(19) United States
(12) Patent Application Publication
(10) Pub. No.: US 2015/0149940 A1
(43) Pub. Date: May 28, 2015

(54) Medical Test Result Presentation

(71) Applicant: General Electric Company, Schenectady, NY (US)

(72) Inventors: Willi Kaiser, Freiburg (DE); Rolf Band, Freiburg (DE); Klaus May, Freiburg (DE)

(73) Assignee: GENERAL ELECTRIC COMPANY, Schenectady, NY (US)

(21) Appl. No.: 14/092,538

(22) Filed: Nov. 27, 2013

(51) Int. Cl. A61B 5/00; A61B 5/0205; A61B 5/16; G06F 3/0484

(52) U.S. Cl. CPC A61B 5/7435; G06F 3/04842; A61B 5/0205; A61B 5/16
USPC 715/765

(57) ABSTRACT

Methods of presenting medical test results include a plurality of test interpretation statements automatedly determined from medical test data. Each automatedly determined test interpretation statement includes a specificity value and a reliability value. A normalized quality is calculated for each of the plurality of test interpretation statements. A graphical display is operated to present a graphical user interface that includes a visual representation of the normalized quality of the plurality of test interpretation statements. A test interpretation statement is presented on the graphical user interface after a selection associated with the visual representations of the normalized quality of the plurality of test interpretation statements.

### BACKGROUND

The present disclosure is related to the field of medical data presentation. More specifically, the present disclosure is related to the presentation of automated medical test interpretation.

Computerized electrocardiographic (ECG) interpretation has become widely accepted in the medical field. Physicians frequently utilize this technique as a back-up to their own interpretation of ECG results, or as a check to ensure that abnormal ECG waveform pathologies have not been overlooked. The interpretation of ECG waveforms is difficult and even physicians may be misled due to the complexity of the analysis that must be performed. In many instances, multiple test or algorithms must utilized to obtain a conclusive results as the results of a single test may fail to distinguish correctly between healthy and pathological ECGs or between different ECG pathology.

Exercise tests utilizing a treadmill or a stationary bicycle have increased in popularity as a useful diagnostic tool of cardiac health. One advantage of an exercise test over a resting ECG test is the increased number of physiological measurement values that may be obtained as the body is put under a stress and then recovers from that stress. These physiological measurement values have the power to predict morbidity/mortality rates, coronary artery disease, and also can analyze the functional response of a patient to exercise. Ideally, a physician would take all of these physiological measurements from the exercise stress test and compare the measurement to the known limits for each of these values as determined by scientific experiments to come to a complete assessment of the patient's health as determined by the exercise test.

Due to recent increases in the number of useful physiological measurement values and applicable analysis algorithms and limits it has become very difficult for a physician to know and apply everything that is needed for a complete assessment of the exercise test. Additionally, it is increasingly difficult for a physician to understand a meaning of an algorithm result and to identify pathologies that are identified with combinational algorithms that compare limits of multiple measurement values.

Previous solutions have sought to convert exercise test assessments into a series of textual statements. However, the clinician is left to make diagnosis and treatment decisions from these statements without additional guidance or information to facilitate evaluation and/or comparison between textual statements.

### BRIEF DISCLOSURE

An exemplary embodiment of a method of presenting medical test results includes a plurality of test interpretation statements which are automatedly determined from medical test data. Each test interpretation statement includes a specificity value and a reliability value. A normalized quality for each of the plurality of test interpretation statements is calculated. A graphical display is operated to present a graphical user interface (GUI) comprising a visual representation of a normalized quality of the plurality of test interpretation statements.

An exemplary embodiment of a medical presentation of exercise test interpretation results includes a plurality of test interpretation statements that are automatedly determined from exercise test data. Each test interpretation statement includes a specificity value and a reliability value. A normalized quality for each of the plurality of test interpretation statements is calculated from the specificity value and a reliability value. The plurality of exercise test interpretation statements are separated into at least a risk category, a functional response category and an ischemia category. Embodiments may also include an overall or summary category. It is to be recognized that the risk category may represent the patient's risk of cardiovascular disease (CVD) risk. In some embodiments, this may include sudden cardiac death (SCD) risk, but the disclosure of the risk category is not so limited.

A visual representation of the risk category is presented with a first GUI object associated with the calculated normalized quality of the plurality of test interpretation statements in the risk category. A visual representation of the functional response category is presented with a second GUI object associated with the calculated normalized quality of the plurality of test interpretation statements in the functional response category. A visual representation of the ischemia category is presented with a third GUI object associated with the calculated normalized quality of the plurality of test interpretation statements in the ischemia category. An input selecting one of the first, second, or third GUI objects is received. The graphical display is operated to present the plurality of test interpretation statements of the risk, functional response, or ischemia category based upon the received input selection.

An embodiment of a computer readable medium includes computer readable code that upon execution by the processor causes the processor to carry out a series of functions. A graphical user interface (GUI) that includes a plurality of categories of exercise test interpretations is presented on a graphical display. A plurality of exercise test interpretations are received. Each exercise test interpretation includes a quality score. A plurality of GUI objects are presented on a graphical display. A GUI object of the plurality is associated to each of the plurality of categories of exercise test interpretations in the GUI. Each of the GUI objects represents a maximum quality score for the exercise test interpretations in each of the categories of exercise test interpretation. An input selecting one of the plurality of categories of exercise test interpretations is received. At least one exercise test interpretations of the selected category of exercise test interpretations is presented in the GUI on the graphical display.

### BRIEF DESCRIPTION OF THE DRAWINGS

FIG. 1 is a schematic diagram of a system for presenting medical test results.

FIG. 2 is a flow chart that depicts an exemplary embodiment of a method of presenting medical test results.

FIGS. 3A and 3B depict exemplary embodiments of graphical user interfaces.

FIGS. 4A-D depict alternative exemplary embodiments of graphical user interfaces

FIG. 5A-D depict still further embodiments of graphical user interfaces.

### DETAILED DISCLOSURE

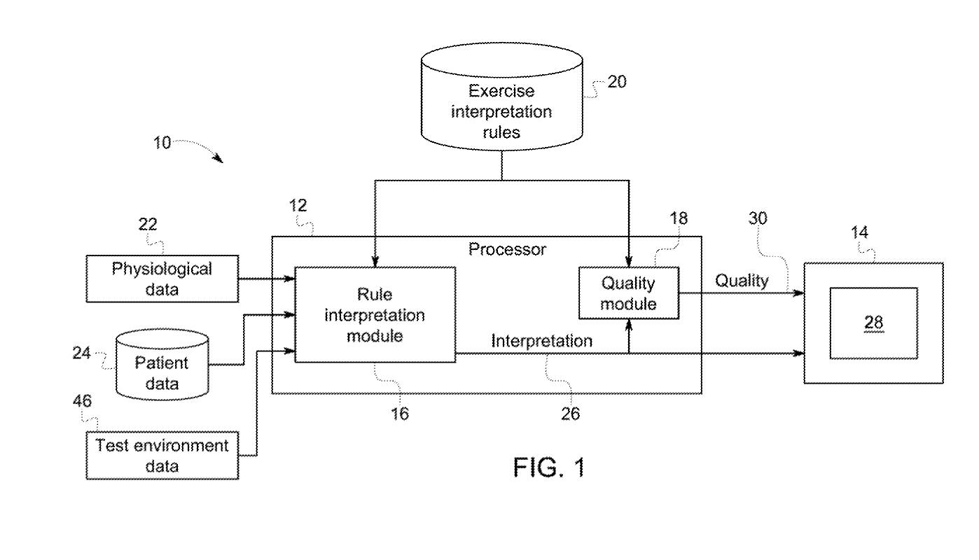

FIG. 1 depicts an exemplary embodiment of a system 10 for carrying out a method of presenting medical test results. The system 10 generally includes a processor 12 and a graphical display 14. The processor 12 is configured to access and execute computer readable code exemplarily in the form of software or software modules. Such computer readable code may be stored integrally to the processor 12 or may be stored at a location physically apart from the processor but communicatively connected to the processor 12. The exemplary embodiment of the system 10 depicted in FIG. 1 depicts two modules, a rule interpretation module 16 and a quality module 18. It is to be understood that while these modules are depicted as being separate, in embodiments, these modules may be combined together and achieve the same functionality. Similarly, although the embodiment of the system 10 depicts a single processor that executes both rule interpretation module 16 and quality module 18 it is to be understood that references to “a processor” contemplate and include distributed processing implementations that divide the execution of the rule interpretation module and the quality module 18 between multiple processors.

In embodiments, the processors are communicatively connected to a database of exercise interpretation rules 20. The rules and/or information stored therein are used in execution of the rule interpretation module 16 and quality module 18 by the processor 12. The system 10 further includes physiological data 22 which in the exemplary embodiments as described in further detail herein is physiological data acquired from a patient through the course of an exercise test. Such exercise test data generally may include, but is not limited to, electrocardiogram (ECG) data, blood pressure, test duration, and current workload (e.g. METS), but may also include any other forms of physiological data as will be recognized as may be acquired in the performance of an exercise test. Patient data 24 which may include previously stored patient data, exemplarily as part of a patient electronic medical record (EMR) or additional data or patient information entered by a clinician before, during, or after the stress test can be used by the rule interpretation module 16 as described in further detail herein. In still further embodiments, test environment data 46, which may include an identification of the test device (e.g. treadmill, bicycle, etc.) or a test type (e.g. Bruce protocol, etc.) may be used by the rule interpretation module 16 as described in further detail herein.

As will be described in greater detail herein, the system 10 operates to produce at least one automated interpretation of the physiological data of the exercise test and operate the graphical display 14 to present the interpretation in a GUI 26 on the graphical display 14. Additionally, a quality for the interpretation statement is calculated and used by the GUI in the presentation of the interpretation statement.

In an exemplary embodiment, the patient data 24 may include standard patient demographical, physiological, or medical information as may be stored in an EMR. This may include a patients age, gender, race, height, and weight, recent lab results or diagnosis (e.g. high cholesterol, diabetes, angina) and may further include information such as current medication (e.g. beta blockers). In still further embodiments, the patient data 24 may include an indication of the type of test being performed, the specific test equipment, and/or test duration. Additional information regarding the medical test can facilitate the selection of exercise test interpretation rules from the database 20.

As the patient undergoes the exercise test, physiological data 22 is acquired and may be temporality stored. The physiological data 22 is provided to the rule interpretation module 16 being implicated by the processor 12. The physiological data 22 may include ECG data such as five lead or a twelve lead, ECG measurements, but may also include a variety of calculated values representing additional physiological measurements. These calculated values may also include information processed from the recorded ECG waveforms. This processed information may include ST depression, detection of arrhythmia, the direction of the ST/HR loop, heart rate recovery, and an MET value.

As stated above, the exercise interpretation rule database 20 includes a plurality of physiological measurement limits or ranges that have been deemed to be correlated to a particular pathology. The rules may include Boolean statements which include one or more physiological measurement limit statements or other patient data. In an exemplary embodiment, the exercise interpretation rules 20 are divided into categories, as will be described in greater detail herein. In an risk prediction category, rules such as a DTS of less than −10 or heart rate recovery of less than twelve beats per minute (BPM), indicate a risk of morbidity or mortality by cardiovascular disease (CVD). T-wave alternans greater than or equal to 65 microvolts are indicative of an increased risk or malignant arrhythmias. In the group of rules for determining cardiac functional response, embodiments may include a rule such as if MET's are less than or equal to five, then that patient has an insufficient exercise capacity. Alternatively, if the heart rate used is less than or equal to 0.8, then the patient is experiencing chronotropic incompetence. Exercise interpretation rules in the category for identifying coronary artery disease or ischemia may include an ST depression of greater than or equal to 1 millimeter, an ST/HR slope of greater than or equal to 2.4 microvolts per BPM, an ST/HR lop that is counterclockwise, or ST/HR hysteresis that is greater than or equal to 0.25 millimeters. These exercise interpretation rules are applied by the rule interpretation module 16 to the physiological data 22 and patient data 24 in order to produce a plurality of interpretation statements 26. The interpretations statements 26 are presented in a graphical user interface (GUI) 28 presented on a graphical display 14.

Additionally, the interpretation statements 26 are further provided to the quality module 18, which, as described herein in further detail, determines a quality for each of the interpretation statements. The determined quality is then used as described herein to modify the presentation of the interpretation statements in the GUI 28. In general, the quality module 18 uses a specificity for each of the interpretation statements and a reliability for each of the interpretation statements in order to calculate a quality value 30 representative of the quality of the interpretation statement. One or more of these quality values further are visually presented in the GUI 28 as described herein.

In an exemplary embodiment, the specificity is a statistical analysis of correlation between the exercise interpretation rules that resulted in the interpretation statement and the category within which the interpretation rule/interpretation statement is categorized. In an embodiment, the specificity is defined by the equation: TN/(TN+FP), wherein TN is the number of true negatives and FP is the number of false positives. In a non-limiting embodiment, values for TN and FP can be obtained by a comparison between each of the interpretation statements with clinical references, which may be located in one or more databases of patient outcomes and medical records. In each of the databases, non-identifying patient information that includes stress test results and an outcome of patient health. In merely exemplary embodiments, each of the non-identifying patient information may include time of death and reason for death, and may also indicate evidence of vessel stenosis, myocardial infarction, diabetes, etc. A specificity of the interpretation statement categorized in the ischemia category is reflective of the correlation or conditionality between the physiological data meeting the exercise interpretation rule that results in the interpretation statement and a diagnosis of ischemia.

In exemplary embodiments, the reliability is an estimation of the quality of the physiological data that is used to meet the exercise interpretation rule. Physiological data that contain a large number of artifacts, discontinuities, or noise may exemplarily be determined as having a lower reliability than higher quality physiological data. Alternatively, it may be known that sometimes the interpretation and/or a specific rule is susceptible to the identification of false positives and this may results in a lower reliability. In still further embodiments, the exercise interpretation rules may further rely upon patient data in addition to the physiological data and in some cases if the patient data is missing, while the rule may still be applied, this may result in a lower reliability for that interpretation statement. In still further embodiments, there may be something in the patient data that reflects a decrease reliability in a specific interpretation rule/statement. Non-limiting embodiments may include interpretation rules/statements that may have a set, or weight as a secondary factor. In a still further embodiment, a patient's occupational status may further adjust a reliability. In a non-limiting example of such an adjustment, a reduced heart rate response to the exercise test may be a reduced reliability if it is identified that the patient does not work or is engaged in a sedentary lifestyle.

The quality module 18 may calculate the quality value 30 as a normalized output based upon the specificity and the reliability. In an exemplary embodiment, the quality value may be calculated as a product of the specificity and the reliability. In still other embodiments, the specificity and reliability may be combined in more complex manners and the quality value may be defined by a series of thresholds and resulting quality values reflective of characteristics of individual interpretation rules or interpretation statements. It is understood that in some embodiments, a single interpretation statement may result from multiple rules. In such a case, each rule may have a different quality value depending upon the manners in which the individual rules define the criteria for the interpretation statement.

Table 1 below presents merely exemplary embodiment of categorized interpretation statements, and merely exemplary of specificities, reliabilities, and a resulting normalized quality values for the interpretation statements.

| | | | |
|---|---|---|---|
| | | Reli- | Normal- |
| | | ability | ized |
| | Speci- | (estima- | Quality |
| Statement | ficity | tion) | Value |

Risk

| | | | |
|---|---|---|---|
| Probable increased risk of cardiovascular | 0.858 | 1 | 86 |
| event | | | |
| Probable risk of cardiovascular event | 0.763 | 0.8 | 45 |
| (FVE recovery) | | | |
| Probable increased risk of malignant | 0.943 | 0.8 | 75 |
| arrhythmias | | | |
| Probable increased risk of | 0.963 | 0.8 | 80 |
| stroke/cardiovascular event | | | |
| Exercise induced bundle branch block | 0.996 | 0.5 | 58 |
| Exercise induced wide QRS tachycardia | 0.996 | 0.5 | 55 |
| Exercise induced atrial fibrillation | 1 | 0.5 | 49 |
| Exercise induced supraventricular | 0.996 | 0.5 | 52 |
| tachycardia | | | |
| Undefined risk | | | −17 |

Functional response

| | | | |
|---|---|---|---|
| Significantly reduced heart rate response | 0.893 | 0.9 | 81 |
| to exercise | | | |
| Reduced heart rate response to exercise | 0.712 | 0.7 | 50 |
| Insufficient exercise capacity | 0.944 | 0.9 | 85 |
| Reduced exercise capacity | 0.811 | 0.7 | 55 |
| Abnormal blood pressure response | 0.983 | 0.8 | 77 |
| Insufficient rate pressure response | 0.936 | 0.8 | 75 |
| Undefined functional response | | | −17 |

Ischemia (CAD)

| | | | |
|---|---|---|---|
| ST/T changes indicative of ischemia | 0.964 | 1 | 96 |
| (ST/HR hysteresis) | | | |
| ST/T changes indicative of ischemia | 0.899 | 1 | 90 |
| (ST/HR hysteresis + HR reserve used) | | | |
| ST/T changes indicative of ischemia | 0.896 | 1 | 88 |
| (ST/HR index + HR reserve used) | | | |
| ST/T changes may be clinically | 0.801 | 0.65 | 50 |
| significant (ST peak) | | | |
| ST/T changes may be clinically | 0.746 | 0.6 | 45 |
| significant (ST recovery) | | | |
| Cannot rule out clinically significant ST/T | 0.687 | 0.6 | 40 |
| changes | | | |

Others

| | |
|---|---|
| Probably normal exercise response | 17 |
| Undefined exercise response | −17 |
| No ECG | −20 |

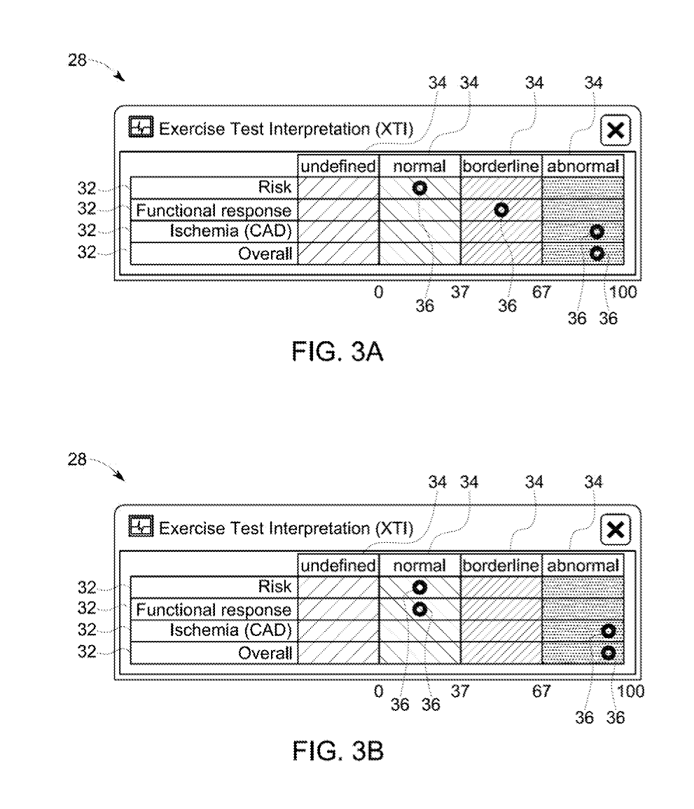

FIGS. 3-5 depict exemplary embodiments of the presentation of the interpretation statements 26 and the quality values 30 in the GUI 28. These examples will be described in further detail herein.

In one particularly advantageous embodiment of the systems and methods as disclosed herein, the GUI presents the interpretation statement and quality value in a manner such as to facilitate the clinician's ability in interpreting the patient's development of ischemia. Ischemia is a restriction in blood supply to tissue which may result in a shortage of oxygen to the tissue. While ischemia is a diagnosis, ischemia can also be due to some other underlying medical condition or other factors, such as, but not limited to coronary artery disease (CAD). In general, an exercise test interpretation with an indication of ischemia results in the clinician referring the patient for a percutaneous cardiac intervention (PCI) which requires catheterization of the patient in order to diagnosis the ischemia and verify or rule out coronary artery disease. PCI is expensive and invasive and therefore, presentation of an assessment of the patient's condition in an ischemia category in conjunction with an indication of the patient's condition in a functional response category may enable the clinician to discriminate between those patients wherein the ischemia is also experiencing an impaired functional response, which is a further indication of coronary artery disease, from those patients wherein the identified ischemia does not result in an impaired functional response, which is a sign that the ischemia has low impact and might be treated with a less aggressive or non-invasive response, exemplarily monitoring or an adjustment of medication.

FIGS. 3A and 3B depict exemplary embodiments of GUIs 28 which may be presented by the graphical display 14 (FIG. 1). Looking first to FIG. 3A, the GUI 28 is presented as a table that defines a plurality of exercise test interpretation categories 32, exemplarily “risk,” “functional response,” “ischemia,” and “overall.” The GUI 28 further identifies a plurality of columns that identify generalized assessments of the patient condition within each category. Exemplarily, these assessments 34 are denoted “undefined”, “normal”, “borderline”, and “abnormal.” In an exemplary embodiment, the “undefined” assessment is an indication that the physiological data was somehow insufficient to produce an assessment of that category of conditions within that exercise interpretation. In an still further embodiment, the normal, borderline, and abnormal assessments can further include relative numerical values representative of the quality values as described herein associated with each of the assessments. In a still further embodiment the normalized numerical scales can be further associated with a color gradation, exemplarily from green to yellow to red also reflective of the assessment. Each category 32 includes a GUI object 36 that is representative of a quality value associated with that category of interpretation. In an exemplary embodiment, the GUI object 36 is indicative of the maximum quality value for the interpretation statements in each category. In an exemplary embodiment, the GUI object 36 associated with the overall category is reflective of the maximum quality value for any interpretation statement in the exercise test results. In a still further embodiment, the GUI object 36 is indicative of an average or weighted average of the quality values for the interpretation statement for that category.

Comparatively looking at FIGS. 3A and 3B, the GUI 28 in FIG. 3A depicts an exemplary exercise test interpretation wherein an abnormal assessment of ischemia is found with a borderline assessment of functional response. Such a patient may exemplarily be identified by the clinician as requiring further assessment with PCI. To the contrary, in the GUI 28 of FIG. 3B the abnormal ischemia assessment is paired with a normal functional response assessment which may be indicative to the clinician that the PCI test is not necessary at this time and a less aggressive approach, exemplarily monitoring or a follow up checkup in six months may be prescribed rather than the PCI test. The embodiments of the GUI 28 depicted in 3A and 3B provide categorical assessments of the patient's exercise test interpretation results, and such relative categorical results can facilitate the clinician's ability to act upon that information without the clinician having to sort through a plurality of interpretation statements or assessments in order to make sense of an overall exercise test.

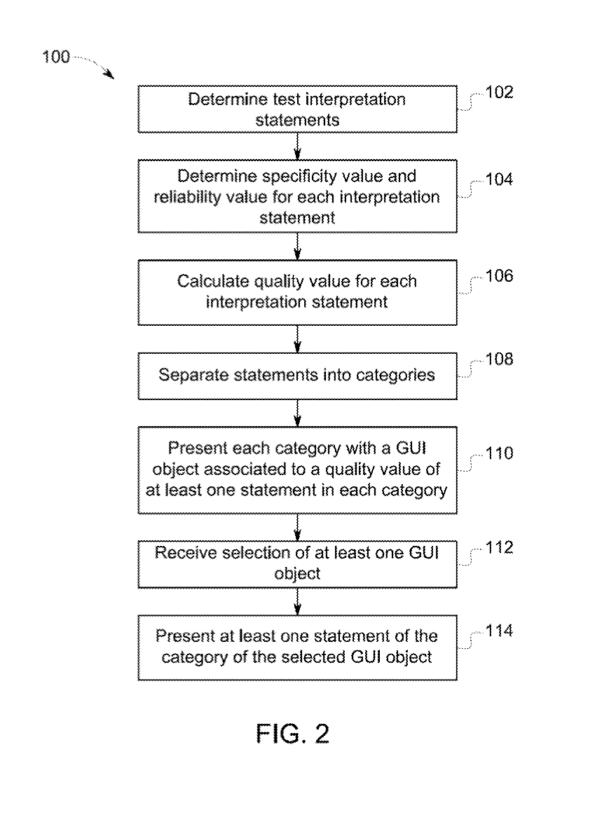

FIG. 2 is a flow chart that depicts an exemplary embodiment of a method 100 of presenting medical test results. The method 100 begins at 102 wherein test interpretation statements are determined from at least physiological data acquired by a medical test, exemplarily an exercise test. As described above, the test interpretation statements are determined by the application of a plurality of exercise interpretation rules to the physiological data. In an exemplary embodiment, this results in a plurality of interpretation statements. Each interpretation statement is associated with at least one reasoning text, as will be described in further detail herein.

An exercise test interpretation may include any number of interpretation statements from any of a plurality of statement categories, exemplarily as described herein as risk, functional response, and ischemia; however, it will be recognized that in alternative embodiments other categories may be used. Each of the plurality of exercise interpretation rules represents a pathological condition resulting in an abnormal or borderline exercise test. Each of these exercise rules may include value limits and/or ranges for physiological data values or may comprise a Boolean statement combining one or more values and/or value ranges or limits.

The fulfillment of an exercise interpretation rule results in an identification of an associated interpretation statement. In an exemplary embodiment, the rule that was fulfilled in order to figure out the identification of the interpretation statement is identified as the associated reasoning text. In some embodiments, it will be understood that a single interpretation statement may be supported by a plurality of reasoning texts if two or more exercise interpretation rules identifying the same interpretation statement are fulfilled by the physiological data.

Next, at 104 a specificity value and a reliability value is determined for each of the interpretation statements. In an exemplary embodiment, the specificity value may be stored along with the exercise interpretation rule and may be a statistical analysis of the correlation between that exercise interpretation rule and the assessment of the health of the patient in the category to which the specific interpretation statement is a part. As exemplarily described above, the specificity may be a ratio of true negatives to the sum of true negatives and false positives. The reliability value may be determined upon the available physiological data and/or patient data, or as described above based upon specific information found in the patient data.

At 106, the specificity value and the reliability value are used to calculate a quality value for each interpretation statement. The quality value may exemplarily be a normalized value, such as on a scale of 0 to 100 with 100 being a highly reliable indication of abnormality while 0 indicates no abnormality, yet rules for a borderline or abnormal patient condition in an exercise test interpretation category were fulfilled. As described above, the calculation of the quality value may be a product of the specificity value and the reliability value, or may be another calculation or quantification based upon the specificity value and reliability value.

At 108, the interpretation statements are separated into categories. As previously disclosed in the exemplary embodiments used herein, the categories are risk, functional response, and ischemia, while other categories will be recognized by a person of ordinary skill in the art. In an exemplary embodiment, the interpretation statement may have been previously sorted into categories, exemplary by sorting the exercise interpretation rules into categories within which the interpretation statement stays while in other embodiments only those interpretation statements that have been determined for the current patient are analyzed and separated into the categories.

At 110, a GUI is presented that presents each category with a GUI object that is associated to a quality value of at least one statement in each category. As described above, the GUI object may be associated to the maximum quality value associated to an interpretation statement in each of the categories, while in alternative embodiments, the GUI object may be associated to an average or weighted average of the quality values of the interpretation statements in each category. Exemplary embodiments of the GUI 28 are described above with respect to FIGS. 3A and 3B.

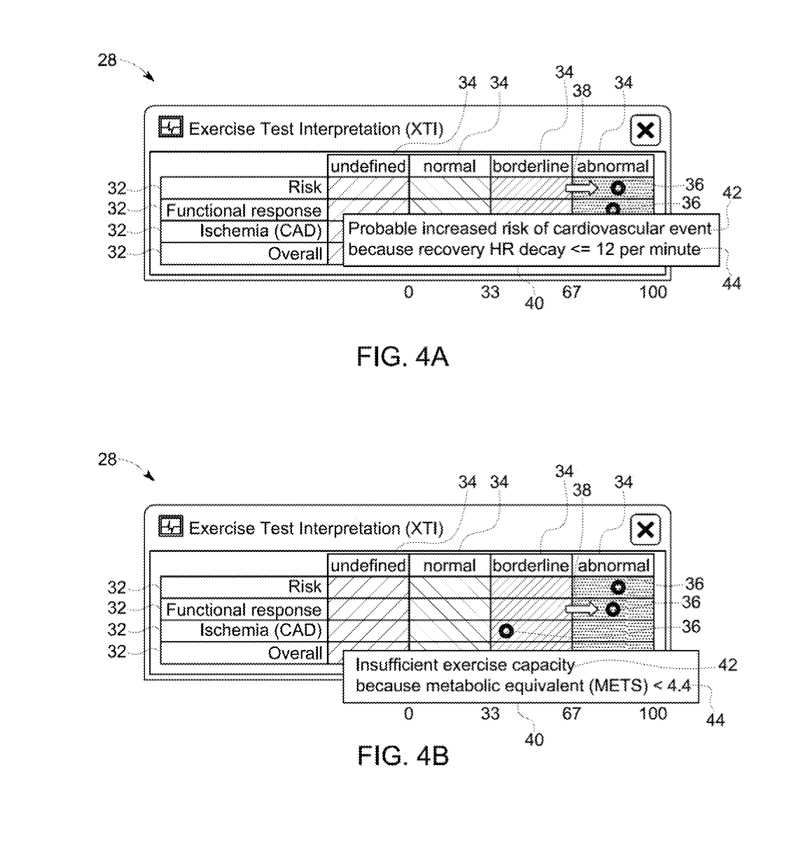

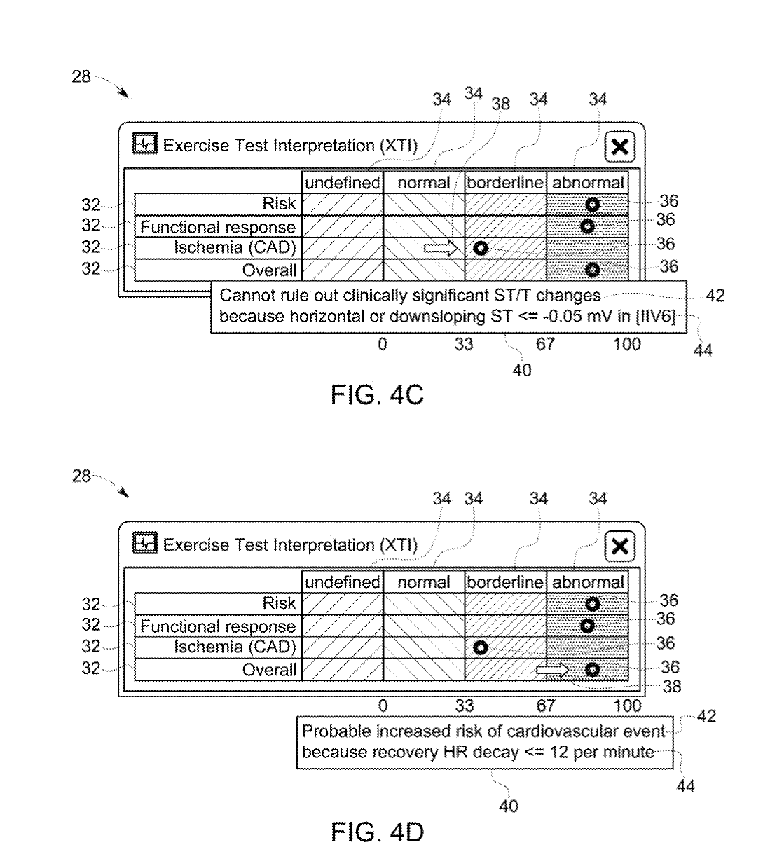

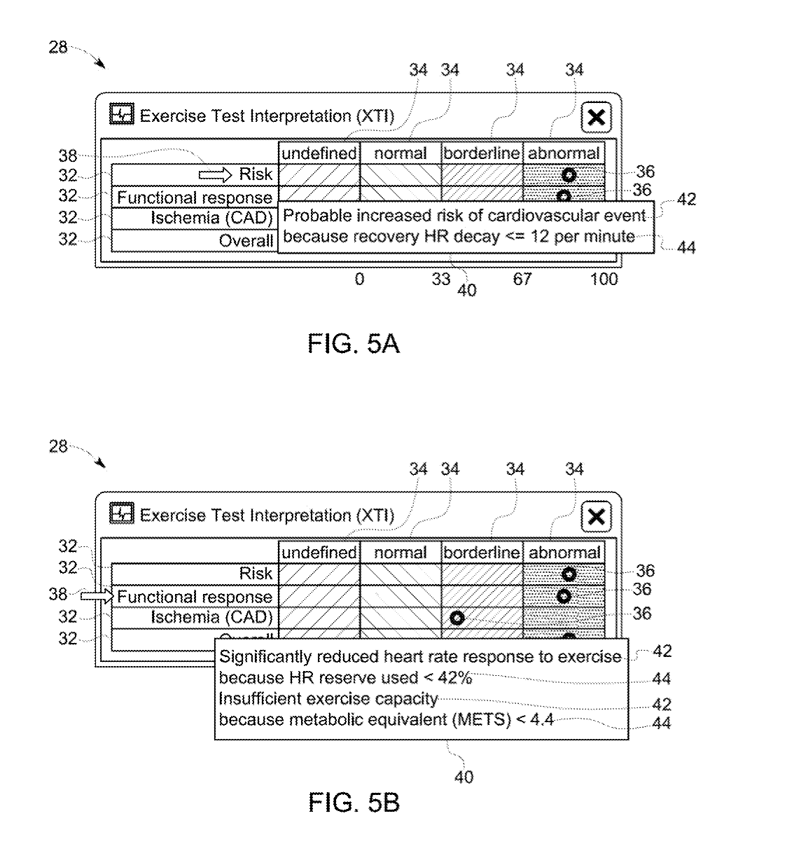

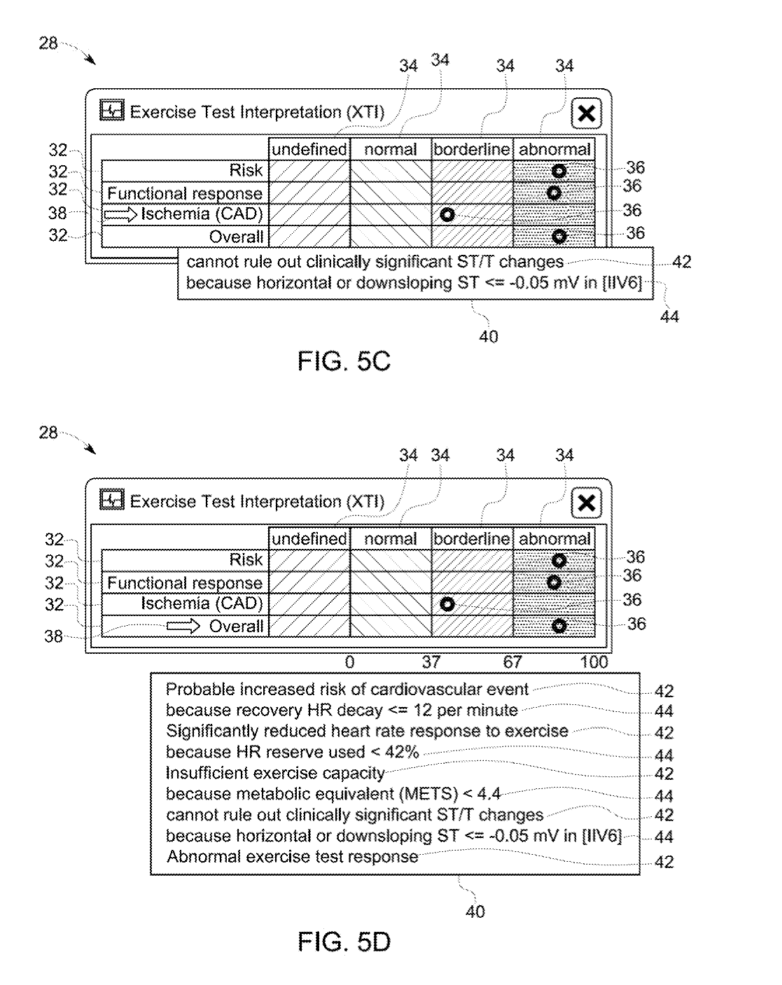

At 112, a selection of at least one GUI object is received. As will be described in further detail herein with respect to FIGS. 4 and 5, a selection of at least one GUI object may exemplarily be a touch input at a GUI object or may be a cursor position or selection of a GUI object. In exemplary embodiments, the GUI object selected may further include an identification of a category 32 in the GUI 28. As will be described in further detail herein at 114, at least one interpretation statement of the category of the selected GUI object is presented upon the receipt of the at least one GUI object at 112. Two different exemplary embodiments of presentation schemes will be described in further detail herein with respect to FIGS. 4A-4D and 5A-5D. In exemplary embodiments, the at least one interpretation statement is presented in a pop-up box in the GUI; however, this is not intended to be limiting on the scope of the manners in which the at least one interpretation statement may be presented, as alternative embodiments of the GUI may include a dedicated text field for the presentation of the interpretation statement.

FIGS. 4A-4D depicts an exemplary embodiment of a GUI 28 as disclosed herein. In FIGS. 4A-4D, the arrow 38 represents a cursor or touch input in selecting a GUI object 36. The exemplary embodiments of the GUI 28 depicted in FIGS. 4A-4D exemplarily have the GUI objects 36 located at a maximum quality value associated with one of the interpretation statements in the exercise test interpretation category 32. The selection of the GUI object 36 results in a pop-up box 40 that presents both the interpretation statement associated with that maximum quality value represented by the GUI object 36 as well as a reasoning text 44 that supports the exercise interpretation statement 42 as described above. These are exemplarily depicted in each of 4A-4D for each of the categories 32 of exercise test interpretation. Further to the description above regarding the overall category 32, it is to be noted that the GUI object 36 for the overall category is reflective of the position of the GUI object 36 for the risk category 32 as this is the highest quality value of any of the interpretation statements. Similarly, selection of the GUI object 36 associated with the overall category 32 results in presentation of the same interpretation statement 42 and reasoning text 44 in 4D as presented in 4A when the GUI object 36 associated with the risk category is selected.

FIGS. 5A-5D depict an alternative embodiment, wherein selection of a GUI object indicative of the category 32 exemplified by the arrow 38 results in a pop-up window 40 in the GUI 28 that presents all of the interpretation statements 42 and reasoning texts 44 associated with that category. This distinction can exemplarily be seen by a comparison between the GUI 28 in FIG. A and the GUI 28 in FIG. 5B. In FIG. 5B, the functional response category includes two interpretation statements 42, and therefore, selection of the functional response category 32 results in presentation of both of the interpretation statements 42 and both of the associated reasoning texts 44. This is still further highlighted in FIG. 5D wherein selection of the overall category 32 results in a pop-up window 40 that presents all of the interpretation statements 42 and associated reasoning texts 44 determined for the entire exercise test interpretation. In an alternative embodiment, selection of the “overall” category results in presentation of the interpretation statements 42 and associated reasoning text 44 from each of the “risk,” “functional response,” and “ischemia” categories.

It is to be recognized that the GUI embodiments depicted in FIGS. 4A-D and 5A-D are merely exemplary of embodiments of the GUI as may be used within the scope of the present disclosure and some embodiments of user inputs and resulting responses, although a person of ordinary skill in the art will recognize that features of both of these embodiments may be used in conjunction or other modifications may be made while still being within the scope of the present disclosure.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of presenting medical test results, the method comprising:
automatedly determining a plurality of test interpretation statements from medical test data, each test interpretation statement having a specificity value and a reliability value;
calculating a normalized quality for each of the plurality of test interpretation statements; and
operating a graphical display to present a graphical user interface (GUI) comprising a visual representation of the normalized quality of the plurality of test interpretation statements.

2. The method of claim 1, further comprising, separating the plurality of exercise test interpretation statements into at least a first category and a second category.

3. The method of claim 2, wherein the visual representation of the normalized quality of the plurality of test interpretation statements comprises:
presenting a visual representation of the first category with a first GUI object associated with the first category, the first GUI object indicative of the calculated normalized quality of the plurality of test interpretation statements in the first category; and
presenting a visual representation of the second category with a second GUI object associated with the second category, the second GUI object indicative of the calculated normalized quality for each of the plurality of test interpretation statements in the second category.

4. The method of claim 3, further comprising:
receiving an input selecting one of the first GUI object or second GUI object; and
operating the graphical display to present at least one of the test interpretation statements of the plurality of test interpretation statements of the first category or the second category based upon the received input selection.

5. The method of claim 4, wherein the first GUI object is indicative of a maximum calculated normalized quality of the plurality of test interpretation statements in the first category and the second GUI object is indicative of a maximum calculated normalized quality of the plurality of test interpretation statements in the second category.

6. The method of claim 4, wherein the plurality of exercise test interpretation statements are separated into at least a first category, a second category, and a third category, and further comprising:
presenting a visual representation of the third category with a third GUI object associated with the third category, the third GUI object indicative of a maximum calculated normalized quality of the plurality of test interpretation statements in the third category.

7. The method of claim 6, further comprising:
presenting a visual representation of a summary with a fourth GUI object associated with the summary, the fourth GUI object indicative of a maximum calculated normalized quality of the plurality of test interpretation statements.

8. The method of claim 7, wherein the medical test data comprises physiological data from an exercise test, and the first category is cardiovascular risk, the second category is functional response, and the third category is ischemia.

9. The method of claim 4, wherein the specificity value is a statistical evaluation of a specificity of each interpretation statement, and the normalized quality is a product of the specificity value and the reliability value.

10. The method of claim 1, wherein the visual representation of the normalized quality of the plurality of test interpretation statements comprises a categorical presentation of the normalized quality.

11. A method of presentation of exercise test interpretation results, the method comprising:
automatedly determining a plurality of test interpretation statements from exercise test data, each test interpretation statement having a specificity value and a reliability value;
calculating a normalized quality for each of the plurality of test interpretation statements from the specificity value and the reliability value;
separating the plurality of exercise test interpretation statements into at least a cardiovascular disease risk category, a functional response category, and an ischemia category;
presenting a visual representation of the cardiovascular disease risk category with a first GUI object associated with the calculated normalized quality of the plurality of test interpretation statements in the cardiovascular disease risk category;
presenting a visual representation of the functional response category with a second GUI object associated with the calculated normalized quality of the plurality of test interpretation statements in the functional response category;
presenting a visual representation of the ischemia category with a third GUI object associated with the calculated normalized quality of the plurality of test interpretation statements in the ischemia category,
receiving an input selecting one of the first, second, or third GUI objects; and
operating the graphical display to present at least one test interpretation statement of the plurality of test interpretation statements of the cardiovascular disease risk, functional response, or ischemia category based upon the received input selection.

12. The method of claim 11, wherein the first GUI object is indicative of a maximum calculated normalized quality of the plurality of test interpretation statements in the cardiovascular disease risk category, the second GUI object is indicative of a maximum calculated normalized quality of the plurality of test interpretation statements in the functional response category, and the third GUI object is indicative of a maximum calculated normalized quality of the plurality of test interpretation statements in the ischemia category.

13. The method of claim 12, further comprising:
presenting a visual representation of a summary with a fourth GUI object associated with the summary, the fourth GUI object indicative of a maximum calculated normalized quality of the plurality of test interpretation statements;
wherein upon receiving an input selecting the fourth GUI object, presenting the plurality of test interpretation statements.

14. The method of claim 11, wherein the reliability value is a numerical representation of the quality of the exercise test data used to automatedly determine the test interpretation statement.

15. The method of claim 11, further comprising:
automatedly determining a plurality of reasoning texts based upon the exercise test data, each of the reasoning texts defined by exercise test data, wherein each reasoning text is associated with a test interpretation statement.

16. The method of claim 15, wherein the specificity is a numerical representation of the accuracy of the at least one exercise test condition in supporting the test interpretation statement.

17. A computer readable medium programmed with computer readable code that upon execution by a processor causes the processor to:
present a graphical user interface (GUI) comprising a plurality of categories of exercise test interpretations on a graphical display;
receive a plurality of exercise test interpretations, each exercise test interpretation comprising a quality score;
present a plurality of GUI objects, a GUI object of the plurality associated to each of the plurality of categories of exercise test interpretations in the GUI on the graphical display, wherein each of the GUI objects represents a maximum quality score for exercise test interpretations in each category of exercise test interpretations;
receive an input selecting one of the plurality of categories of exercise test interpretations; and
present at least one exercise test interpretation of the selected category.

18. The computer readable medium of claim 17, wherein the plurality of categories of exercise test interpretations comprise cardiovascular disease risk, functional response, and ischemia.

19. The computer readable medium of claim 17, wherein the input selecting one of the plurality of categories is a selection of the GUI object and the exercise test interpretations are presented in a pop-up window in the GUI presented on the graphical display.

20. The computer readable medium of claim 19, wherein the exercise test interpretations each further comprise at least one reasoning text, the at least one reasoning text presented in the pop-up window in the GUI presented on the graphical display.
